(19) United States
(12) Patent Application Publication
(10) Pub. No.: US 2024/0293101 A1
(43) Pub. Date: Sep. 5, 2024

(54) COMPRESSION PLATE, MAMMOGRAPHY APPARATUS, AND IMAGE CAPTURING SYSTEM

(71) Applicant: FUJIFILM Corporation, Tokyo (JP)

(72) Inventors: Hisatsugu HORIUCHI, Kanagawa (JP); Lisako NOBUYAMA, Kanagawa (JP); Masataka SUGAHARA, Kanagawa (JP); Koji TANINAI, Kanagawa (JP)

(73) Assignee: FUJIFILM Corporation, Tokyo (JP)

(21) Appl. No.: 18/442,058

(22) Filed: Feb. 14, 2024

(30) Foreign Application Priority Data
Mar. 1, 2023 (JP) 2023-031301

(51) Int. Cl. A61B 8/00; A61B 8/08

(52) U.S. Cl. CPC A61B 8/403; A61B 8/0825

(57) ABSTRACT

A compression plate includes a wall surface portion facing a chest wall of an examinee and a bottom surface portion connected to the wall surface portion, and at least a part of the wall surface portion is a low rigidity portion that extends from the bottom surface portion. The low rigidity portion has a lower rigidity than other portions of the compression plate.

### CROSS-REFERENCE TO RELATED APPLICATION

This application is based on and claims priority under 35 USC 119 from Japanese Patent Application No. 2023-031301 filed on Mar. 1, 2023, the disclosure of which is incorporated by reference herein.

### BACKGROUND OF THE INVENTION

### 1. Field of the Invention

The present disclosure relates to a compression plate, a mammography apparatus, and an image capturing system.

### 2. Description of the Related Art

A mammography apparatus is known that irradiates the breast of an examinee with radiation emitted from a radiation source and detects the radiation transmitted through the breast with a radiation detector to capture a radiation image.

For example, JP2014-124364A discloses a compression plate for capturing an image while compressing an examinee during imaging using a mammography apparatus. In this compression plate, a gap member made of a material softer than the material of the support body is provided inside a slit provided in each wall portion.

Further, an ultrasonography apparatus is known that captures an ultrasound image of the breast by causing an ultrasound probe to perform scanning along the breast of the examinee to scan the breast with ultrasound.

The presence or absence of a tumor may be detected by comparing the above-mentioned breast radiation image and ultrasound image. In particular, there are many cases in which a tumor, a calcification, or the like is suspected on a chest wall side of an examinee, and it is necessary to ensure that no ultrasound examination including the chest wall is omitted.

However, because the shape of the breast changes depending on the presence or absence of compression, it is difficult to compare the position of the tumor between mammography and ultrasound. For this reason, there is a method in which an ultrasound probe is mounted on a compression plate and an ultrasound examination is performed in a compressed state.

### SUMMARY OF THE INVENTION

In the above-described method, the position of the chest wall surface of the compression plate, that is, the surface facing the chest wall of the examinee, is a position where the chest wall side of the examinee can be imaged with X-rays as wide as possible. However, at this position, it is difficult to perform an ultrasound examination of the chest wall since the ultrasound probe on the compression plate cannot be brought into close contact with the chest wall of the examinee. In addition, for example, even in a case in which an attempt is made to bring the ultrasound probe into close contact with the examinee from the outside of the compression plate, it is not possible to perform an ultrasound examination of the chest wall due to there not being a sufficient space.

On the other hand, it is conceivable to move the position of the chest wall surface of the compression plate toward the chest wall side of the examinee from the current position in order to improve the accessibility of the ultrasound probe to the chest wall. However, in a case in which the compression plate is moved, the examinee cannot be brought into close contact with an imaging table, and as a result, an imaging range of the mammography apparatus is reduced.

The present disclosure has been made in consideration of the above-mentioned circumstances, and an object of the present disclosure is to provide a compression plate, a mammography apparatus, and an image capturing system that can perform an ultrasound examination on a chest wall side while enabling X-ray imaging without moving a position of a chest wall surface of the compression plate.

In order to achieve the above object, according to a first aspect of the present disclosure, there is provided a compression plate that is used in a mammography apparatus, the compression plate comprising: a wall surface portion facing a chest wall of an examinee; and a bottom surface portion connected to the wall surface portion, in which at least a part of the wall surface portion is a low rigidity portion that extends from the bottom surface portion, the low rigidity portion having a lower rigidity than other portions of the compression plate.

According to a second aspect of the present disclosure, in the compression plate according to the first aspect, the low rigidity portion may be made of an elastically deformable member, the low rigidity portion before deformation may be located between an end surface of an imaging unit included in the mammography apparatus and an effective imaging region of the imaging unit, and at least a part of the low rigidity portion after deformation may be movable toward an examinee side from the end surface of the imaging unit.

According to a third aspect of the present disclosure, in the compression plate according to the first aspect, the low rigidity portion may include a part of the bottom surface portion.

According to a fourth aspect of the present disclosure, the compression plate according to the first aspect may further comprise an intermediate member to be interposed between the examinee and an ultrasound probe, and the intermediate member may be provided at at least one of a chest wall surface of the wall surface portion facing the chest wall of the examinee or a surface of the wall surface portion opposite to the chest wall surface.

In order to achieve the above object, according to a fifth aspect of the present disclosure, there is provided a compression plate that is used in a mammography apparatus, the compression plate comprising: a wall surface portion facing a chest wall of an examinee, in which a part of the wall surface portion is open.

In order to achieve the above object, according to a sixth aspect of the present disclosure, there is provided a mammography apparatus comprising: the compression plate according to any one of the first to fifth aspects.

In order to achieve the above object, according to a seventh aspect of the present disclosure, there is provided an image capturing system comprising: an ultrasonography apparatus including an ultrasound probe; and a mammography apparatus including the compression plate according to any one of the first to fifth aspects.

According to the aspects of the present disclosure, it is possible to perform an ultrasound examination on the chest wall side while enabling X-ray imaging without moving the position of the chest wall surface of the compression plate.

### BRIEF DESCRIPTION OF THE DRAWINGS

FIG. 1 is a view showing an example of an image capturing system according to a first embodiment.

FIG. 2 is a side view showing an example of a configuration of a compression unit.

FIG. 3A is a perspective view showing an example of the appearance of a compression plate according to the first embodiment.

FIG. 3B is a cross-sectional view showing a state in which the compression plate shown in FIG. 3A is taken in the center along line IIIB-IIIB.

FIG. 4A is a perspective view showing an example of a low rigidity portion.

FIG. 4B is a perspective view showing an example of the compression plate in a state in which the low rigidity portion is removed.

FIG. 5A is a perspective view showing an example of the appearance of the compression plate and an ultrasound probe according to the first embodiment.

FIG. 5B is a partial cross-sectional view showing a periphery of a wall surface portion of the compression plate shown in FIG. 5A.

FIG. 5C is a partial cross-sectional view showing a state in which the low rigidity portion of the compression plate shown in FIG. 5A is pressed by the ultrasound probe.

FIG. 6A is a view showing a state of the compression plate and the ultrasound probe as viewed from above a head of an examinee.

FIG. 6B is a partially enlarged view showing a state in which the low rigidity portion is pressed by the ultrasound probe shown in FIG. 6A.

FIG. 7A is a view showing a state in which a breast of the examinee is compressed from the side with the compression plate.

FIG. 7B is a partial cross-sectional view showing a state in which the low rigidity portion of the compression plate is pressed by the ultrasound probe shown in FIG. 7A.

FIG. 8A is a view schematically showing an example of a state of the low rigidity portion before deformation.

FIG. 8B is a view schematically showing an example of a state of the low rigidity portion after deformation.

FIG. 9A is a perspective view showing an example of the appearance of a compression plate according to a second embodiment.

FIG. 9B is a partial cross-sectional view showing a periphery of a wall surface portion of the compression plate shown in FIG. 9A.

FIG. 10 is a partial cross-sectional view showing a periphery of a wall surface portion of a compression plate according to a third embodiment.

FIG. 11 is a perspective view showing an example of the appearance of a compression plate according to a fourth embodiment.

### DESCRIPTION OF THE PREFERRED EMBODIMENTS

Embodiments of the present invention will be described in detail below with reference to the drawings. Note that each embodiment does not limit the present invention.

### First Embodiment

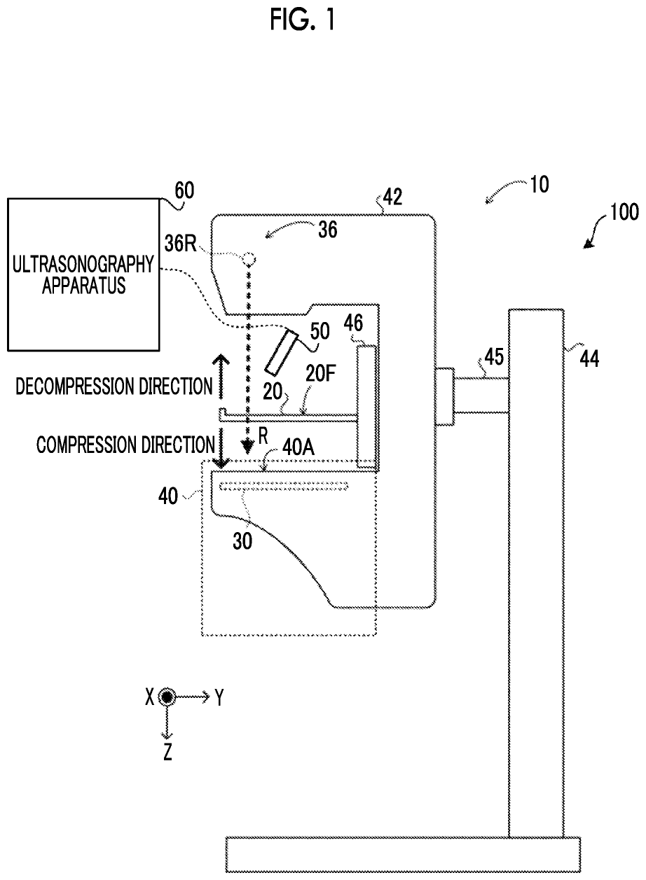

FIG. 1 is a view showing an example of an image capturing system 100 according to a first embodiment. The image capturing system 100 according to the present embodiment comprises a mammography apparatus 10 and an ultrasonography apparatus 60. In FIG. 1, a depth direction is an X-axis direction, a left-right direction is a Y-axis direction, and an up-down direction is a Z-axis direction.

The mammography apparatus 10 according to the present embodiment is an apparatus that uses a breast of the examinee as a subject and captures a radiation image of the breast by irradiating the breast with radiation R (for example, X-rays). Note that the mammography apparatus 10 may be an apparatus that images the breast of the examinee in a state in which the examinee is sitting on a chair (including a wheelchair) or the like (sitting state) in addition to a state in which the examinee is standing (standing state).

As shown in FIG. 1, a mammography apparatus 10 according to the present embodiment comprises a compression plate 20, a radiation detector 30, a radiation emitting unit 36, an imaging table 40, an arm part 42, a base 44, a shaft part 45, and a compression unit. 46. The imaging table 40 is an example of an imaging unit.

The radiation detector 30 detects the radiation R transmitted through the breast of the examinee. The radiation detector 30 is disposed inside the imaging table 40. In the mammography apparatus 10 according to the present embodiment, in a case in which imaging is performed, the breast of the examinee is positioned on an imaging surface 40A of the imaging table 40 by a user such as a doctor or a radiology technician. For example, the imaging surface 40A and the like with which the breast of the examinee comes into contact are made of carbon or the like in terms of the transmittance and intensity of the radiation R.

The radiation detector 30 detects the radiation R transmitted through the breast of the examinee and the imaging table 40, generates a radiation image based on the detected radiation R, and outputs image data that represents the generated radiation image. The type of the radiation detector 30 is not particularly limited. For example, the radiation detector 30 may be an indirect conversion type radiation detector that converts the radiation R into light and converts the converted light into electric charges, or may be a direct conversion type radiation detector that directly converts the radiation R into electric charges.

The radiation emitting unit 36 comprises a radiation source 36R. The radiation emitting unit 36 is provided on the arm part 42 together with the imaging table 40 and the compression unit 46. In addition, the mammography apparatus 10 comprises the arm part 42, the base 44, and the shaft part 45. The arm part 42 is held by the base 44 so as to be movable in the up-down direction (Z-axis direction). The shaft part 45 connects the arm part 42 to the base 44. The compression unit 46 and the arm part 42 can be relatively rotated with respect to the base 44 separately, using the shaft part 45 as a rotation axis. In the present embodiment, gears (not shown) are provided in each of the shaft part 45, the arm part 42, and the compression unit 46. Each gear is switched between an engaged state and a disengaged state to connect each of the arm part 42 and the compression unit 46 to the shaft part 45. One or both of the arm part 42 and the compression unit 46 connected to the shaft part 45 are rotated integrally with the shaft part 45.

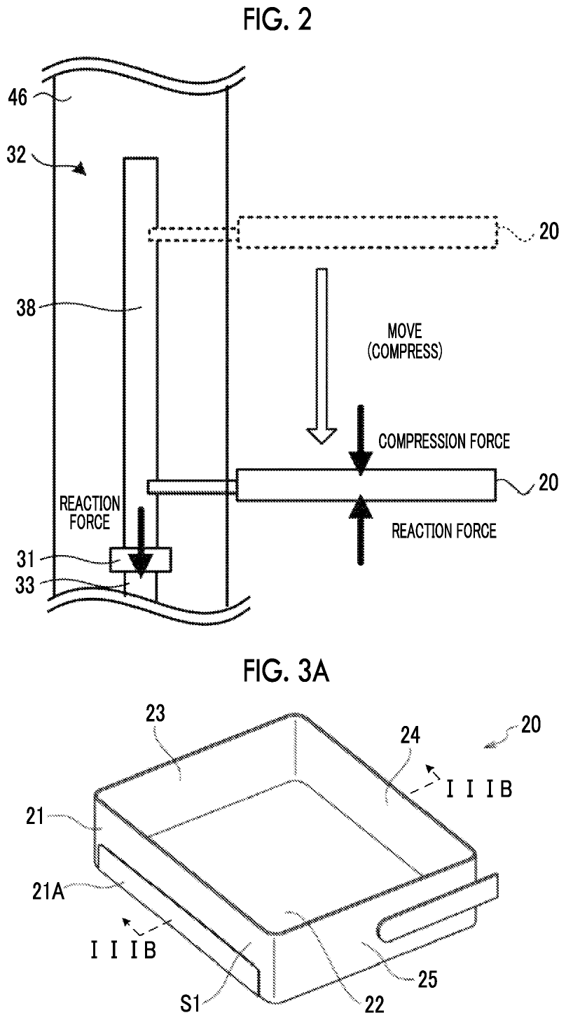

FIG. 2 is a side view showing an example of the configuration of the compression unit 46. Note that FIG. 2 shows a side view of the compression unit 46 when viewed in a direction opposite to that of FIG. 1.

As shown in FIG. 2, a compression plate drive part 32, a compression force detection sensor 33, and the compression plate 20 are provided in the compression unit 46. The compression unit 46 comprises the compression plate drive part 32 including a motor 31 and a ball screw 38 and the compression force detection sensor 33. The compression force detection sensor 33 has a function of detecting the compression force by detecting the reaction force to the compression force that compresses the entire breast by the compression plate 20.

The compression plate 20 according to the present embodiment is a compression member, and is moved in the up-down direction (Z-axis direction) by the compression plate drive part 32 to compress the breast of the examinee between the compression plate 20 and the imaging table 40. As shown in FIG. 1, for the movement direction of the compression plate 20, the direction in which the breast is compressed, in other words, the direction in which the compression plate 20 becomes closer to the imaging surface 40A is referred to as a “compression direction” and the direction in which the compression of the breast is released, in other words, the direction in which the compression plate 20 becomes closer to the radiation emitting unit 36 is referred to as a “decompression direction”.

It is preferable that the compression plate 20 is optically transparent in order to check positioning or a compressed state in the compression of the breast. In addition, the compression plate 20 is made of a material having high transmittance for the radiation R. Further, it is desirable that the compression plate 20 is made of a material that facilitates the transmission of ultrasonic waves from an ultrasound probe 50 of the ultrasonography apparatus 60. Examples of the material forming the compression plate 20 include resins such as polymethylpentene, polycarbonate, acrylic, or polyethylene terephthalate. In particular, polymethylpentene is suitable as the material forming the compression plate 20 since it has low rigidity, high elasticity, and high flexibility and has suitable values for acoustic impedance that affects the reflectance of ultrasonic waves and an attenuation coefficient that affects the attenuation of ultrasonic waves.

Note that the compression plate 20 is not limited to one that compresses the entire breast, but may be one that compresses a part of the breast. In other words, the compression plate 20 may be smaller than the breast. As such a compression plate 20, for example, a compression plate 20 used for so-called spot imaging, in which a radiation image is captured of only a region where a lesion exists, is known.

On the other hand, the ultrasonography apparatus 60 is an apparatus that includes the ultrasound probe 50 and is used to capture an ultrasound image of the breast of the examinee as a subject by a user.

The ultrasound probe 50 is moved along a surface 20F (see FIG. 1, a surface opposite to the surface that comes into contact with the breast of the examinee) of the compression plate 20 by the user and scans the breast with ultrasound to acquire an ultrasound image of the breast.

The ultrasound probe 50 comprises a plurality of ultrasound transducers which are one-dimensionally or two-dimensionally arranged. Each of the ultrasound transducers transmits ultrasonic waves based on an applied drive signal, receives ultrasound echoes, and outputs a received signal.

Each of the plurality of ultrasound transducers is composed of, for example, a transducer in which electrodes are formed at both ends of a piezoelectric material (piezoelectric body) such as a piezoelectric ceramic represented by lead (Pb) zirconate titanate (PZT) or a polymer piezoelectric element represented by polyvinylidene difluoride (PVDF). In a case in which a pulsed or continuous wave drive signal is transmitted to apply a voltage to the electrodes of the transducer, the piezoelectric body is expanded and contracted. Pulsed or continuous ultrasonic waves are generated from each transducer by the expansion and contraction and the ultrasonic waves are combined to form an ultrasound beam. Further, each transducer receives the propagated ultrasonic waves and is then expanded and contracted to generate an electric signal. The electric signal is output as an ultrasound received signal and is input to the main body of the ultrasonography apparatus 60 through a cable indicated by a dotted line.

Incidentally, in a case in which the compression plate 20 is disposed at a position suitable for X-ray imaging by the mammography apparatus 10, the ultrasound probe 50 cannot be brought into close contact with the chest wall of the examinee. Therefore, it is difficult to perform an ultrasound examination of the chest wall.

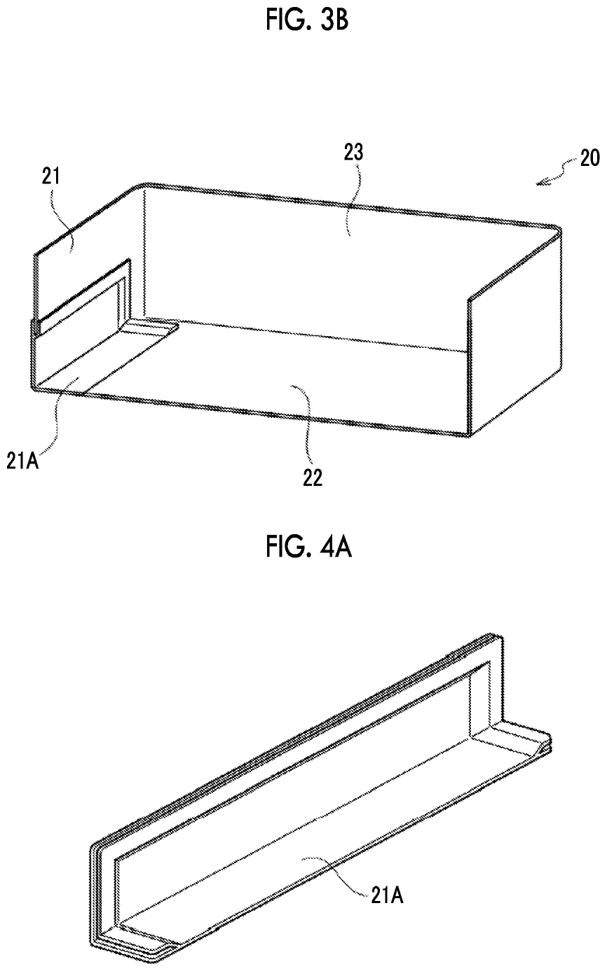

The compression plate 20 according to the present embodiment has a deformable portion from the bottom surface of the compression plate 20 to the chest wall surface, as shown in FIGS. 3A and 3B, for example. Thereby, only the portion pressed by the ultrasound probe 50 during the ultrasound examination can be moved from the position of X-ray imaging to the chest wall side of the examinee. Therefore, it is possible to perform an ultrasound examination by bringing the ultrasound probe 50 into close contact with the chest wall of the examinee.

FIG. 3A is a perspective view showing an example of the appearance of the compression plate 20 according to the first embodiment. FIG. 3B is a cross-sectional view showing a state in which the compression plate 20 shown in FIG. 3A is taken in the center along line IIIB-IIIB.

As shown in FIGS. 3A and 3B, the compression plate 20 according to the present embodiment comprises a wall surface portion 21 facing the chest wall of the examinee and a bottom surface portion 22 connected to the wall surface portion 21. A part of the wall surface portion 21 is a low rigidity portion 21A extending from the bottom surface portion 22. The low rigidity portion 21A is a portion that has a lower rigidity than the rigidity of other portions of the compression plate 20 and is deformable by the pressing of the ultrasound probe 50. The low rigidity portion 21A includes, for example, a part of the bottom surface portion 22. Note that the surface of the wall surface portion 21 facing the chest wall of the examinee is defined as a chest wall surface S1.

Side wall portions 23, 24, and 25 are provided around the bottom surface portion 22. That is, the compression plate 20 is a box-shaped member in which the wall surface portion 21, the side wall portions 23, 24, and 25 are provided around the bottom surface portion 22 and the upper portion thereof is open. The ultrasound probe 50 can be accessed through an upper opening of the compression plate 20. Note that a structure may be employed in which the side wall portions 23, 24, and 25 are not provided.

The low rigidity portion 21A continuously extends from a part of the bottom surface portion 22. Therefore, it is possible to reduce artifacts caused by the seam of the low rigidity portion 21A in a case in which an ultrasound examination is performed on a portion having a high degree of interest in the vicinity of the chest wall of the examinee.

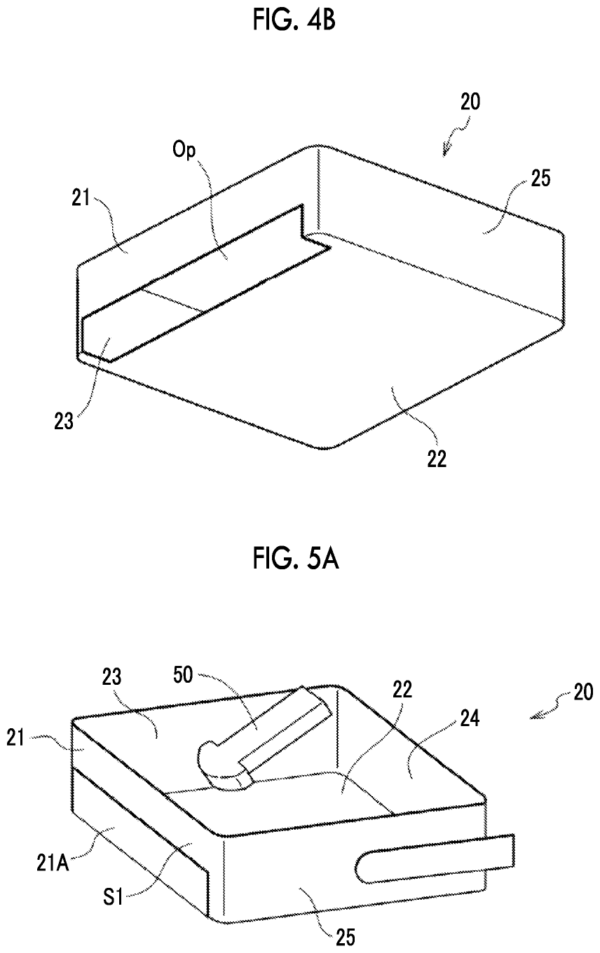

FIG. 4A is a perspective view showing an example of the low rigidity portion 21A. FIG. 4B is a perspective view showing an example of the compression plate 20 in a state in which the low rigidity portion 21A is removed.

The low rigidity portion 21A shown in FIG. 4A is fixed in alignment with an opening Op of the compression plate 20 shown in FIG. 4B. A fixing method is not particularly limited, but a method capable of ensuring a sufficient strength is desirable. The low rigidity portion 21A is made of an elastically deformable member. As a material of the low rigidity portion 21A, for example, a rubber member, such as silicon, urethane, or nitrile rubber (NBR), or a foamed foam, such as an elastomer, foamed urethane, foamed acrylic, or foamed polyethylene, is used. On the other hand, the other portions of the compression plate 20 are made of, for example, resins such as polymethylpentene, polycarbonate, acrylic, or polyethylene terephthalate, as described above. That is, the rigidity of the low rigidity portion 21A is lower than the rigidity of other portions of the compression plate 20.

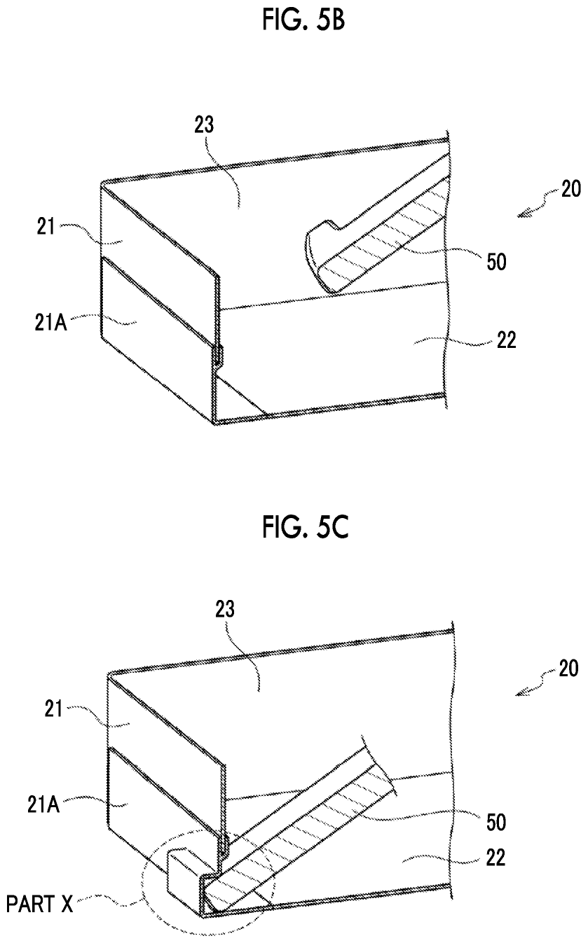

FIG. 5A is a perspective view showing an example of the appearance of the compression plate 20 and the ultrasound probe 50 according to the first embodiment. FIG. 5B is a partial cross-sectional view showing the periphery of the wall surface portion of the compression plate 20 shown in FIG. 5A. FIG. 5C is a partial cross-sectional view showing a state in which the low rigidity portion 21A of the compression plate 20 shown in FIG. 5A is pressed by the ultrasound probe 50.

As shown in part X of FIG. 5C, in a case in which the user presses the low rigidity portion 21A with the ultrasound probe 50, the pressed portion is extended, and the ultrasound probe 50 is brought into close contact with the chest wall of the examinee via the low rigidity portion 21A, and thus an ultrasound examination of the chest wall can be performed.

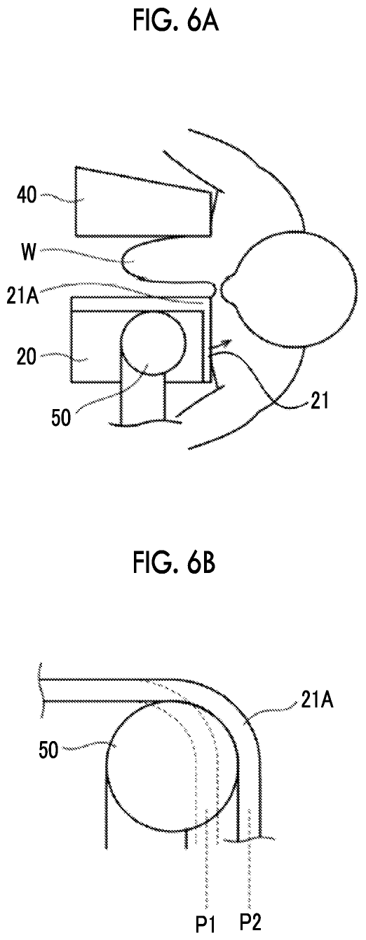

FIG. 6A is a view showing a state of the compression plate 20 and the ultrasound probe 50 as viewed from above the head of the examinee. FIG. 6B is a partially enlarged view showing a state in which the low rigidity portion 21A is pressed by the ultrasound probe 50 shown in FIG. 6A.

As shown in FIG. 6A, the wall surface portion 21 of the compression plate 20 is pressed against the chest wall of the examinee to place a breast W of the examinee between the compression plate 20 and the imaging table 40, and the breast W is compressed by the compression plate 20.

As shown in FIG. 6B, in a state in which the low rigidity portion 21A is not pressed by the ultrasound probe 50, the low rigidity portion 21A is located at a position P1, and the chest wall of the examinee is located at a position corresponding to the position P1. X-ray imaging is performed at this position P1. On the other hand, in a state in which the low rigidity portion 21A is pressed by the ultrasound probe 50, the low rigidity portion 21A extends from the position P1 to a position P2, and the chest wall of the examinee is located at a position corresponding to the position P2. Since the ultrasound examination is performed at this position P2, it is possible to perform the ultrasound examination while compressing the chest wall as compared with the position P1.

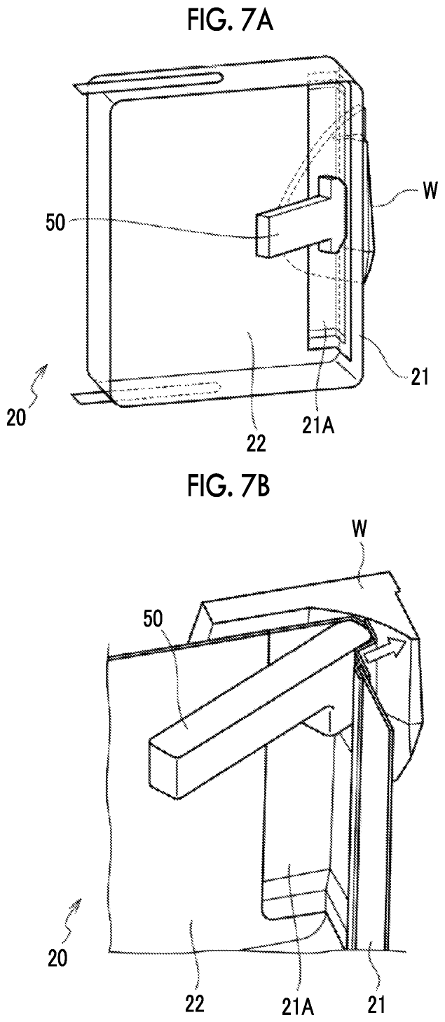

FIG. 7A is a view showing a state in which the breast W of the examinee is compressed from the side with the compression plate 20. FIG. 7B is a partial cross-sectional view showing a state in which the low rigidity portion of the compression plate is pressed by the ultrasound probe 50 shown in FIG. 7A.

As shown in FIGS. 7A and 7B, the breast W of the examinee can be compressed from the side with the compression plate 20. That is, it is also possible to perform ultrasound imaging in a direction in which the wall surface portion 21 of the compression plate 20 is substantially orthogonal to the chest wall of the examinee, that is, in a direction in which the bottom surface portion 22, is pressed against the chest wall of the examinee.

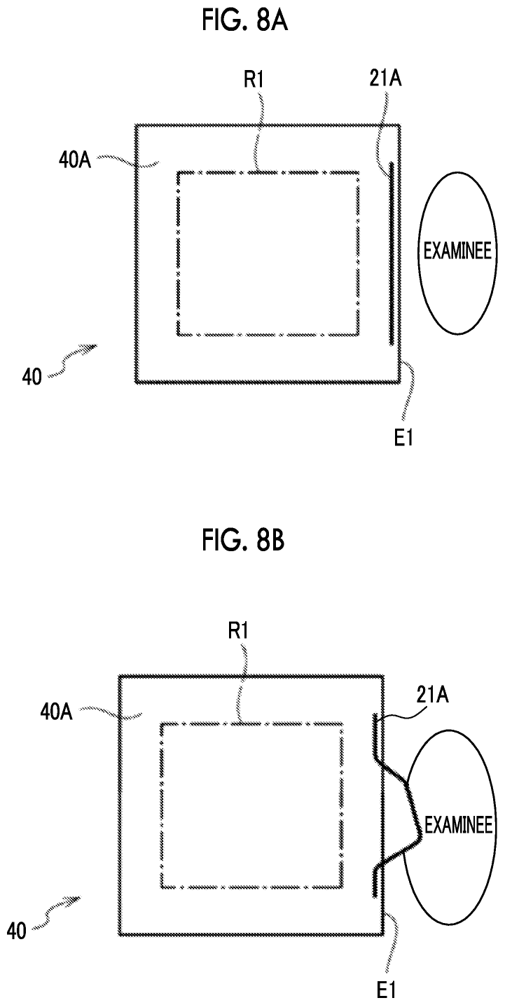

FIG. 8A is a view schematically showing an example of a state of the low rigidity portion 21A before deformation. FIG. 8B is a view schematically showing an example of a state of the low rigidity portion 21A after deformation.

As shown in FIG. 8A, the low rigidity portion 21A before deformation is located between an end surface E1 of the imaging table 40 included in the mammography apparatus 10 and an effective imaging region R1 of the imaging table 40. On the other hand, at least a part of the low rigidity portion 21A after deformation is movable toward the examinee side from the end surface E1 of the imaging table 40, as shown in FIG. 8B.

In X-ray imaging, as shown in FIG. 8A, the optimal position of the low rigidity portion 21A (that is, the wall surface portion 21) of the compression plate 20 is outside the effective imaging region R1 and inside the end surface E1. On the other hand, in ultrasound imaging, as shown in FIG. 8B, the low rigidity portion 21A is moved closer to the examinee side than the end surface E1. In this way, by partially moving the position of the low rigidity portion 21A, during X-ray imaging, it is possible to perform X-ray imaging over a wider region, and during ultrasound imaging, it is possible to perform imaging while compressing the chest wall of the examinee.

In this way, according to the present embodiment, since at least a part of the chest wall surface of the compression plate is deformable, only the portion pressed by the ultrasound probe during the ultrasound examination can be moved from the position of X-ray imaging to the chest wall side of the examinee. Therefore, it is possible to perform an ultrasound examination by bringing the ultrasound probe into close contact with the chest wall of the examinee.

### Second Embodiment

In a second embodiment, an aspect in which the entire wall surface portion of the compression plate is configured as a low rigidity portion will be described.

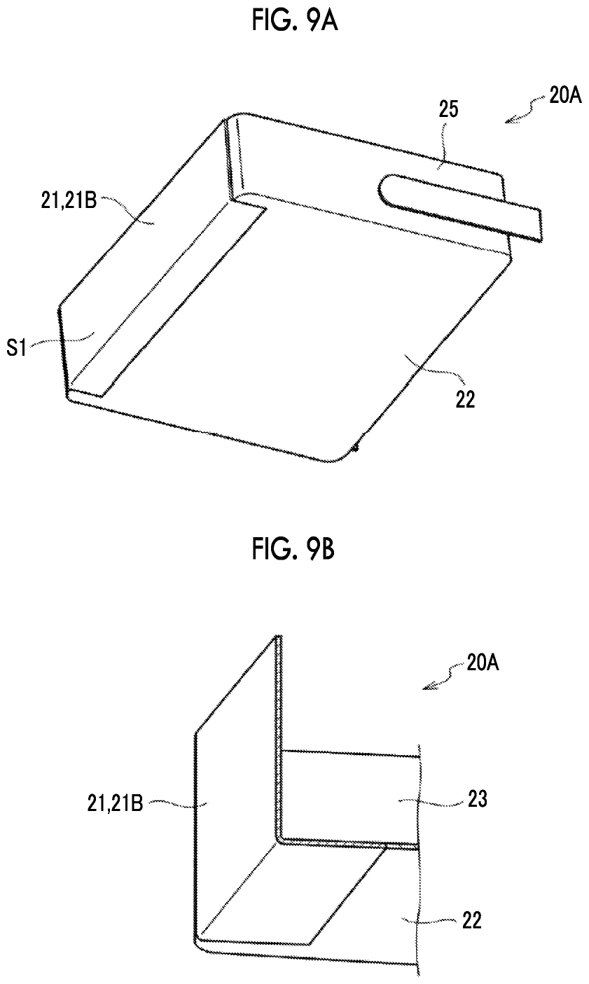

FIG. 9A is a perspective view showing an example of the appearance of a compression plate 20A according to the second embodiment. FIG. 9B is a partial cross-sectional view showing a periphery of a wall surface portion of the compression plate 20A shown in FIG. 9A.

As shown in FIGS. 9A and 9B, in the compression plate 20A according to the present embodiment, the entire wall surface portion 21 is configured as a low rigidity portion 21B. That is, the low rigidity portion 21B extends from the bottom surface portion 22 and constitutes the entire wall surface portion 21. Also in the present embodiment, the low rigidity portion 21B may include a part of the bottom surface portion 22.

In this way, according to the present embodiment, the entire chest wall surface of the compression plate can be deformed. Accordingly, it is possible to perform an ultrasound examination over a wider range.

### Third Embodiment

In a third embodiment, an aspect in which an intermediate member such as a gel sheet is provided on the wall surface portion of the compression plate will be described.

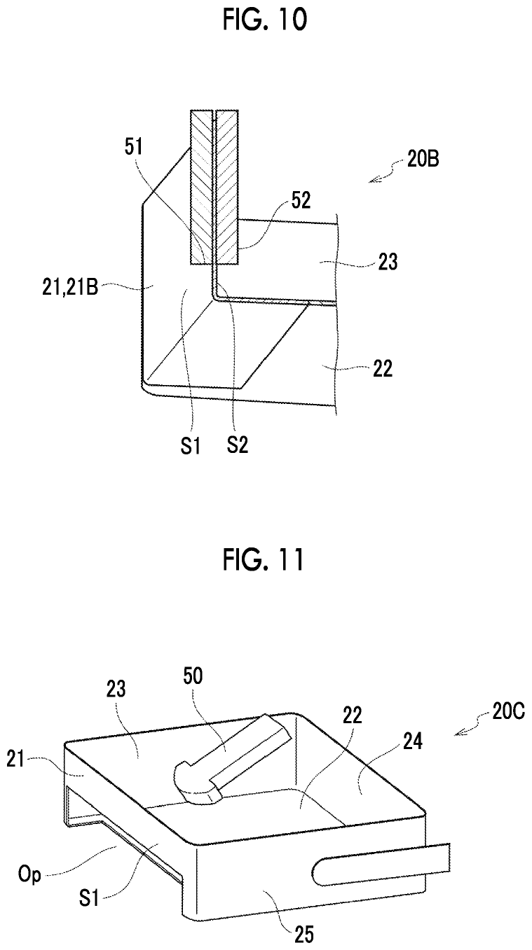

FIG. 10 is a partial cross-sectional view showing a periphery of a wall surface portion of a compression plate 20B according to the third embodiment.

As shown in FIG. 10, the compression plate 20B according to the present embodiment may comprise intermediate members 51 and 52 to be interposed between the breast W of the examinee and the ultrasound probe 50. The intermediate members 51 and 52 are provided on at least one of the chest wall surface S1 of the wall surface portion 21 (the low rigidity portion 21B or the low rigidity portion 21A) or an opposite surface S2 which is a surface of the wall surface portion 21 opposite to the chest wall surface S1. In the example of FIG. 11, the intermediate members 51 and 52 are provided on both the chest wall surface S1 and the opposite surface S2, but they may be provided on only one of the surfaces.

For the intermediate members 51 and 52, for example, a gel sheet or the like in which a gel-like (or jelly-like) acoustic matching material is stored is used. The acoustic matching material referred to here is a member that fills the space between the low rigidity portion 21B and the ultrasound probe 50, fills the space between the examinee and the low rigidity portion 21B, and acoustically couples with the examinee. The acoustic matching material is preferably a material having an acoustic impedance close to that of the examinee, the low rigidity portion 21B, and the ultrasound probe 50. By providing the intermediate member 51, it is possible to improve the adhesiveness between the breast W of the examinee and the compression plate 20B. On the other hand, by providing the intermediate member 52, it is possible to improve the adhesiveness between the compression plate 20B and the ultrasound probe 50.

In this way, according to the present embodiment, an intermediate member such as a gel sheet is provided on the wall surface portion of the compression plate. Accordingly, it is possible to improve the adhesiveness between the breast of the examinee and the compression plate or the adhesiveness between the compression plate and the ultrasound probe.

### Fourth Embodiment

In a fourth embodiment, an aspect in which a part of a wall surface portion of a compression plate is an opening will be described.

FIG. 11 is a perspective view showing an example of the appearance of a compression plate 20C according to the fourth embodiment.

As shown in FIG. 11, the compression plate 20C according to the present embodiment comprises the wall surface portion 21 and a part of the wall surface portion 21 is open. That is, an opening Op is provided in a part of the wall surface portion 21. Further, a part of the bottom surface portion 22 may also be an opening, and a part of the wall surface portion 21 and a part of the bottom surface portion 22 may constitute the opening Op.

Since the compression plate 20C according to the present embodiment is provided with the opening Op instead of the low rigidity portion 21A described above, the ultrasound probe 50 can be directly brought into close contact with the breast W of the examinee. Since the low rigidity portion 21A is not used, it is possible to perform a more precise ultrasound examination including the chest wall.

In this way, according to the present embodiment, the opening is provided in the wall surface portion of the compression plate instead of the low rigidity portion. Therefore, the ultrasound probe can be directly brought into close contact with the breast of the examinee.

In addition, the configurations and operations of the compression plate 20, the mammography apparatus 10, and the image capturing system 100 described in each of the above-described embodiments are merely examples and it goes without saying that they can be changed according to the situation without departing from the gist of the present invention.

Regarding the above embodiments, the following supplementary notes are further disclosed.

### Supplementary Note 1

A compression plate that is used in a mammography apparatus, the compression plate comprising:
a wall surface portion facing a chest wall of an examinee; and
a bottom surface portion connected to the wall surface portion,
in which at least a part of the wall surface portion is a low rigidity portion that extends from the bottom surface portion, the low rigidity portion having a lower rigidity than other portions of the compression plate.

### Supplementary Note 2

The compression plate according to Supplementary Note 1, wherein:
the low rigidity portion is made of an elastically deformable member,
the low rigidity portion before deformation is located between an end surface of an imaging unit included in the mammography apparatus and an effective imaging region of the imaging unit, and
at least a part of the low rigidity portion after deformation is movable toward an examinee side from the end surface of the imaging unit.

### Supplementary Note 3

The compression plate according to Supplementary Note 1 or 2,
in which the low rigidity portion includes a part of the bottom surface portion.

### Supplementary Note 4

The compression plate according to any one of Supplementary Notes 1 to 3, further comprising:
an intermediate member to be interposed between the examinee and an ultrasound probe,
in which the intermediate member is provided at at least one of a chest wall surface of the wall surface portion facing the chest wall of the examinee or a surface of the wall surface portion opposite to the chest wall surface.

### Supplementary Note 5

A compression plate that is used in a mammography apparatus, the compression plate comprising:
a wall surface portion facing a chest wall of an examinee,
in which a part of the wall surface portion is open.

### Supplementary Note 6

A mammography apparatus comprising the compression plate according to any one of Supplementary Notes 1 to 5.

### Supplementary Note 7

An image capturing system comprising:
an ultrasonography apparatus including an ultrasound probe; and
a mammography apparatus including the compression plate according to any one of Supplementary Notes 1 to 5.

What is claimed is:

1. A compression plate that is used in a mammography apparatus, the compression plate comprising:
a wall surface portion facing a chest wall of an examinee; and
a bottom surface portion connected to the wall surface portion,
wherein at least a part of the wall surface portion is a low rigidity portion that extends from the bottom surface portion, the low rigidity portion having a lower rigidity than other portions of the compression plate.

2. The compression plate according to claim 1, wherein:
the low rigidity portion is made of an elastically deformable member,
the low rigidity portion before deformation is located between an end surface of an imaging unit included in the mammography apparatus and an effective imaging region of the imaging unit, and
at least a part of the low rigidity portion after deformation is movable toward an examinee side from the end surface of the imaging unit.

3. The compression plate according to claim 1, wherein the low rigidity portion includes a part of the bottom surface portion.

4. The compression plate according to claim 1, further comprising:
an intermediate member to be interposed between the examinee and an ultrasound probe,
wherein the intermediate member is provided at at least one of a chest wall surface of the wall surface portion facing the chest wall of the examinee or a surface of the wall surface portion opposite to the chest wall surface.

5. A compression plate that is used in a mammography apparatus, the compression plate comprising:
a wall surface portion facing a chest wall of an examinee,
wherein a part of the wall surface portion is open.

6. A mammography apparatus comprising the compression plate according to claim 1.

7. A mammography apparatus comprising the compression plate according to claim 2.

8. A mammography apparatus comprising the compression plate according to claim 3.

9. A mammography apparatus comprising the compression plate according to claim 4.

10. A mammography apparatus comprising the compression plate according to claim 5.

11. An image capturing system comprising:
an ultrasonography apparatus including an ultrasound probe; and
a mammography apparatus including the compression plate according to claim 1.

12. An image capturing system comprising:
an ultrasonography apparatus including an ultrasound probe; and
a mammography apparatus including the compression plate according to claim 2.

13. An image capturing system comprising:
an ultrasonography apparatus including an ultrasound probe; and
a mammography apparatus including the compression plate according to claim 3.

14. An image capturing system comprising:
an ultrasonography apparatus including an ultrasound probe; and
a mammography apparatus including the compression plate according to claim 4.

15. An image capturing system comprising:
an ultrasonography apparatus including an ultrasound probe; and
a mammography apparatus including the compression plate according to claim 5.
